(19) United States
(12) Patent Application Publication
(10) Pub. No.: US 2023/0194389 A1
(43) Pub. Date: Jun. 22, 2023

(54) SAMPLE COLLECTION AND DISPOSAL SWAB

(71) Applicant: Abbott Rapid Diagnostics International Unlimited Company, Dublin (IE)

(72) Inventors: Mi Jung Ji, Seoul (KR); Byungjin Kim, Seoul (KR); Jro Lee, Seoul (KR); Sangyong Park, Seoul (KR)

(21) Appl. No.: 17/922,399

(22) PCT Filed: May 7, 2021
(86) PCT No.: PCT/IB2021/000315

§ 371 Date: Oct. 31, 2022

Related U.S. Application Data
(60) Provisional application No. 63/038,307, filed on Jun. 12, 2020.
(60) Provisional application No. 63/021,526, filed on May 7, 2020.

(51) Int. Cl. G01N 1/10; B01L 3/00

(52) U.S. Cl. CPC G01N 1/10; B01L 3/5023; B01L 3/5029; B01L 2200/16; B01L 2200/18; B01L 2200/026; B01L 2300/042; B01L 2300/069; B01L 2300/123; G01N 2001/1025; G01N 2001/1056

(57) ABSTRACT

Provided herein are devices, kits, systems, and methods for collecting samples for analytical analysis and the safe disposal of sample collection devices after their use. The devices, kits, systems, and methods find use, for example, for disposing of biohazard materials by users in settings that may not be equipped with the professional biohazard disposal systems of laboratories, hospitals, and medical clinics.

This application claims priority to U.S. Provisional Pat. Application Serial No. 63/021,526, filed May 7, 2020; and U.S. Provisional Pat. Application Serial No. 63/038,307, filed Jun. 12, 2020, each of which is incorporated herein by reference in its entirety.

### FIELD

Provided herein are devices, kits, systems, and methods for collecting biological samples for analysis and for the safe disposal of sample collection devices after their use. The devices, kits, systems, and methods find use, for example, for providing biological samples for testing and for disposing of biohazard or other hazardous materials by users in settings that may not be equipped with the professional biohazard disposal systems of laboratories, hospitals, and medical clinics.

### BACKGROUND

Biological samples, such as saliva, mucus, and blood, are often tested for the presence of pathogens using a sample collection device to collect the biological sample and an analytical device to test the biological sample. A user collecting a biological sample using a sample collection device is at risk of being contacted by the biological sample during collection and thus the user may be exposed to a biohazard risk. Further, after transfer of biological sample to the analytical device for testing, sample collection devices may retain a portion of the biological sample and thus the used sample collection devices may also be a biohazard risk. Technologies are needed for handling biological samples and for disposing of sample collection devices comprising biological samples that may pose such biohazard risks.

### SUMMARY

Provided herein are devices, kits, systems, and methods for collecting biological samples for analysis and for the safe disposal of sample collection devices after their use. The devices, kits, systems, and methods find use, for example, for providing biological samples for testing and for disposing of biohazard materials by users in settings that may not be equipped with the professional biohazard disposal systems of laboratories, hospitals, and medical clinics. Accordingly, the technology provided herein reduces biohazard risk to users during a sample preparation process for analytical testing.

For example, the emergence of the COVID-19 pandemic has created a need for substantial testing outside of traditional laboratory and hospital environments. Some tests, such as lateral flow-based immunoassays, may be readily conducted at home, in medical clinics, or non-traditional settings by unsophisticated users. Embodiments of the devices, kits, systems, and methods provided herein provide new ways to minimize the exposure risk to users, the community, healthcare workers, and laboratory personnel to dangerous or biohazardous agents such as SARS-CoV-2 during sample preparation and testing.

In some embodiments, the technology provides a sample collection device comprising an extraction tube body comprising a top end, a central portion, and a bottom end. In some embodiments, the top end is configured to accept a top cap; the bottom end is configured to accept a bottom cap; and the bottom end comprises a dropper feature. Thus, in some embodiments, the sample collection device comprises an extraction tube body, a top cap, and a bottom cap, wherein the extraction tube body comprises a bottom end comprising a dropper feature. In some embodiments, the extraction tube body accepts a swab device. In some embodiments, the swab device comprises a swab handle comprising a proximal end and a distal end, and the swab device comprises a swab end at the distal end of the swab handle. In some embodiments, the swab device has a length that is longer than a length that is the length of the central portion plus the length of the top end.

In some embodiments, the swab device comprises a breakable junction. In some embodiments, the extraction tube body comprises a separation component. In some embodiments, the separation component is configured to interact with the breakable junction (e.g., to facilitate breaking the swab device (e.g., the swab handle) at the breakable junction). In some embodiments, the extraction tube body comprises a fill line. In some embodiments, the fill line marks a volume of approximately 300 µL. In some embodiments, the swab device is flexible. In some embodiments, the swab device can be bent and/or compressed to fit entirely inside said extraction tube body. In some embodiments, the extraction tube body comprises a top cap sealing said top end and a bottom cap sealing said bottom end and at least a part of said swab device is sealed inside said extraction tube body. In some embodiments, the extraction tube body comprises a top cap sealing said top end said dropper feature is exposed. In some embodiments, the dropper feature outputs droplets of approximately 50 µl. In some embodiments, the extraction tube body is made from polyethylene (e.g., high density polyethylene).

In some embodiments, the central portion has a length that is approximately 80.1 mm. In some embodiments, the swab device and/or the swab handle has a length that is greater than approximately 80.1 mm. In some embodiments, the swab device and/or the swab handle has a length that is approximately 71 to 100 mm. In some embodiments, the top end of the extraction tube body has a length that is approximately 7.8 mm or approximately 9.6 mm. In some embodiments, the extraction tube body has an outer diameter that is approximately 10.5 mm. In some embodiments, the extraction tube body has an inner diameter that is approximately 9 mm. In some embodiments, the bottom end of the extraction tube body has a length that is approximately 13 mm. In some embodiments, the dropper feature has an outer diameter that is approximately 2.5 mm. In some embodiments, the dropper feature has an inner diameter that is approximately 1.6 mm.

In some embodiments, the technology provides a kit comprising: a) a swab device comprising a swab end and a swab handle; b) a sample collection device comprising an extraction tube body comprising a dropper feature; and c) a buffer solution. In some embodiments, the sample collection device comprises a top cap and a bottom cap. In some embodiments, the extraction tube body comprises a top end configured to accept the top cap; and the extraction tube body comprises a bottom end configured to accept the bottom cap. In some embodiments, the swab device comprises a breakable junction. In some embodiments, the swab handle of the swab device comprises the breakable junction. In some embodiments, the swab handle of the swab device comprises a proximal end and a distal end, the swab handle comprises the swab end at the distal end, and the swab handle comprises the breakable junction between the proximal end of the swab handle and the distal end of the swab handle. In some embodiments, the breakable junction is between approximately 5 to 9 cm from the swab end.

In some embodiments, the technology provides a kit comprising: a) a swab device comprising a swab handle comprising a proximal end and a distal end, a swab end at the distal end of the swab handle, and a breakable junction between the swab end and the proximal end of the swab handle; b) a sample collection device comprising: i) an extraction tube body comprising a dropper feature; ii) a top cap; and iii) a bottom cap; and c) a buffer solution (e.g., provided in a buffer solution bottle). In some embodiments, kits further comprise an analytical assay device, e.g., an analytical assay device configured to detect a dangerous (e.g., biohazard) substance (e.g., a pathogen). See, e.g., Int′l Pat. App. No. PCT/US21/026183, which is incorporated herein by reference. In some embodiments, kits further comprise a biohazard disposal container.

In some embodiments of kits, the extraction tube body accepts said swab device. In some embodiments of kits, the swab device comprises a breakable junction. In some embodiments of kits, the extraction tube body comprises a separation component. In some embodiments of kits, the separation component is configured to break said swab device. In some embodiments of kits, the swab device comprises a breakable junction and said separation component is configured to interact with said breakable junction. In some embodiments of kits, the extraction tube body comprises a fill line. In some embodiments of kits, the fill line marks a volume of approximately 300 µL. In some embodiments of kits, the swab device is flexible. In some embodiments of kits, the swab handle is flexible. In some embodiments of kits, the swab device can be bent or compressed to fit entirely inside said extraction tube body. In some embodiments of kits, the dropper feature outputs droplets of approximately 50 µl. In some embodiments of kits, the sample collection device is made from polyethylene. In some embodiments of kits, the sample collection device is made from high density polyethylene.

In some embodiments, the technology provides a system comprising: a) a swab device comprising a swab end and a swab handle; b) a sample collection device comprising an extraction tube body comprising a dropper feature; and c) a buffer solution. In some embodiments, the sample collection device comprises a top cap and a bottom cap. In some embodiments, the extraction tube body comprises a top end configured to accept the top cap; and the extraction tube body comprises a bottom end configured to accept the bottom cap. In some embodiments, the swab device comprises a breakable junction. In some embodiments, the swab handle of the swab device comprises the breakable junction. In some embodiments, the swab handle of the swab device comprises a proximal end and a distal end, the swab handle comprises the swab end at the distal end, and the swab handle comprises the breakable junction between the proximal end of the swab handle and the distal end of the swab handle. In some embodiments, the breakable junction is between approximately 5 to 9 cm from the swab end.

In some embodiments, the technology provides a system comprising: a) a swab device comprising a swab handle comprising a proximal end and a distal end, a swab end at the distal end of the swab handle, and a breakable junction between the swab end and the proximal end of the swab handle; b) a sample collection device comprising: i) an extraction tube body comprising a dropper feature; ii) a top cap; and iii) a bottom cap; and c) a buffer solution (e.g., provided in a buffer solution bottle). In some embodiments, systems further comprise an analytical assay device, e.g., an analytical assay device configured to detect a dangerous (e.g., biohazard) substance (e.g., a pathogen). See, e.g., Int′l Pat. App. No. PCT/US21/026183, which is incorporated herein by reference. In some embodiments, systems further comprise a biohazard disposal container.

In some embodiments of systems, the extraction tube body accepts said swab device. In some embodiments of systems, the swab device comprises a breakable junction. In some embodiments of systems, the extraction tube body comprises a separation component. In some embodiments of systems, the separation component is configured to break said swab device. In some embodiments of systems, the swab device comprises a breakable junction and said separation component is configured to interact with said breakable junction. In some embodiments of systems, the extraction tube body comprises a fill line. In some embodiments of systems, the fill line marks a volume of approximately 300 µL. In some embodiments of systems, the swab device is flexible. In some embodiments of systems, the swab handle is flexible. In some embodiments of systems, the swab device can be bent or compressed to fit entirely inside said extraction tube body. In some embodiments of systems, the dropper feature outputs droplets of approximately 50 µl. In some embodiments of systems, the sample collection device is made from polyethylene. In some embodiments of systems, the sample collection device is made from high density polyethylene.

In some embodiments, the technology relates to methods. For example, in some embodiments, methods comprise a) providing a kit as described herein; b) adding a buffer solution to the extraction tube body; c) contacting the swab device (e.g., the swab end) with a sample; d) placing the swab device into the extraction tube body with the swab handle (e.g., the proximal end of the swab handle) extending above a top end of the extraction tube body and the swab end positioned in the buffer solution; e) separating the swab end from at least a portion of the swab handle and/or compressing the swab device into the extraction tube body; f) securing the top cap on the top end of the extraction tube body; g) removing the bottom cap from the bottom end of the extraction tube body; and h) dispensing buffer solution containing sample from the dropper feature. In some embodiments, methods further comprise providing an analytical assay device and conducting an analytical assay on the dispensed buffer solution containing sample by applying the dispensed buffer solution containing sample to the analytical assay device. In some embodiments, methods comprise placing the extraction tube body containing the swab end and/or placing the analytical assay device into a biohazard disposal container. In some embodiments, methods comprise contacting a swab device with a sample, said swab device comprising a swab end and a swab handle; placing the swab device into an extraction tube body with the swab handle extending above a top end of the extraction tube body; and separating the swab end from at least a portion of the swab handle by contacting the outside of the extraction tube body. In some embodiments, methods further comprise securing a top cap on a top end of the extraction tube body; removing a bottom cap from the extraction tube body; and dispensing buffer solution containing sample from a dropper feature.

### BRIEF DESCRIPTION OF THE DRAWINGS

These and other features, aspects, and advantages of the present technology will become better understood with regard to the following drawings.

FIGS. 1A-1K show exemplary swab devices and extraction tube bodies as described herein and an exemplary workflow (e.g., embodiments of methods) for employing swab devices and extraction tube bodies as described herein.

FIG. 1A shows a sample collection device (100) comprising an extraction tube body (110) comprising a fill line (111), a top cap (120), and a bottom cap (130); a swab device (200) comprising a swab end (210) and a swab handle (220) comprising a breakable junction (230); and a dropper bottle (300) comprising a dropper bottle cap (320) and a dropper bottle body (310) containing a buffer solution. The distal end (222) of the swab handle (220) comprises the swab end (210) and the proximal end (221) of the swab handle (220) is opposite the swab end (210).

FIG. 1B shows how buffer is added to the extraction tube after the top cap is removed from the extraction tube. The dropper bottle cap (320) is removed from the dropper bottle body (310) and buffer solution (330) is provided (e.g., as drops) into the extraction tube body (110). The extraction tube body (110) comprises a fill line (111) marking a volume of approximately 300 µL from the bottom of the extraction tube body (110). The fill line (111) provides a reference for a user to observe (900) the filling of the bottom of the extraction tube body (110) with an adequate amount of buffer solution (330) to extract at least a portion of a sample from a swab device (e.g., a swab end of a swab device) upon placement of the swab device (e.g., the swab end of the swab device) into the extraction tube body (110). As shown in FIG. 1B, buffer solution (330) is added to the extraction tube body (110) before a sample is added to the extraction tube body (110) and thus eliminates and/or minimizes the risk of splashing pathogen.

FIG. 1C shows the swab device (e.g., after it has been used to collect a sample from a subject (e.g., a subject with COVID-19)) inserted into the extraction tube containing buffer and being swirled. As shown in FIG. 1C, embodiments provide that the swab device (200) is placed into the extraction tube body (110) with the swab end (210) in the buffer solution (330) and the swab is swirled (e.g., by rotating (800) the swab handle (220)) to provide at least a portion of a sample on the swab end (210) into the buffer solution (330).

FIG. 1D shows the swab end in the extraction tube body after the swab device has been cut or broken at the breakable junction (e.g., to make the swab device short enough to be sealed inside the extraction tube body when the top cap is placed on the extraction tube body). As shown in FIG. 1D, in some embodiments, the swab device (200) comprises a breakable junction (230) and the swab handle (220) of the swab device (200) is broken at the breakable junction (230). As shown in FIG. 1D, the swab end (210) remains present in the buffer solution (330) at the bottom of the extraction tube body (110), which eliminates and/or minimizes the risk of splashing and spreading pathogen.

FIG. 1E shows the top cap (120) being added to the extraction tube body (110) to seal the swab end (210) and a portion of the swab handle (220) in the extraction tube body (110). As shown in FIG. 1E, adding the top cap (120) to the extraction tube body (110) seals the swab end (210) within the extraction tube body (110), thus minimizing and/or eliminating the risk of exposing users or other individuals to a pathogen.

FIG. 1F shows the extraction tube body (110) with the swab end (210) and a portion of the swab handle (220) sealed inside the extraction tube body (110) (e.g., by the top cap (120)).

FIG. 1G and FIG. 1H show a swab device (200) comprising a swab end (210) and a compressed and/or bent swab handle (220) that is sealed inside the extraction tube body (110) (e.g., by the top cap (120)). The swab handle (220) may be unbroken or may be a portion of a swab handle after it is cut or broken at the breakable junction.

FIG. 1I shows the bottom cap (130) being removed from the extraction tube body (110) to reveal the dropper feature (103).

FIG. 1J shows the bottom cap removed from the extraction tube body (110) to reveal the dropper feature (103), e.g., a dropper feature (103) configured to provide drops of sample (500) (e.g., in buffer solution (330))) on the sample well of an analytical assay device (600). In some embodiments, the extraction tube body (110) is flexible and squeezing (700) the extraction tube body increases the pressure inside the extraction tube body (110) that pushes the sample (500) (e.g., in buffer solution (330))) out of the dropper feature (103).

FIG. 1K shows the disposal of two components: 1) an extraction tube body (110) containing a swab end (210) and at least a portion of a swab handle (220); and 2) an analytical assay device (600) (e.g., in a biohazard compatible bag). As shown in FIG. 1J, the bottom cap (130) is placed on (e.g., coupled to) the extraction tube body (110) containing the swab end (210) and at least a portion of the swab handle (220). Accordingly, the swab device (200) is sealed inside the sample collection device (100). In particular, the swab end (210) and at least a portion of the swab handle (220) is sealed inside the extraction tube body (110)) by the top cap (120) and the bottom cap (130). The sample collection device (100) (e.g., the extraction tube body (110), top cap (120), and bottom cap (130)) and the swab device (200) (e.g., the swab end (210) and at least a portion of the swab handle (220)) are disposed of in a biohazard compatible bag (999). The analytical assay device (600) may be disposed of in a biohazard compatible bag (999). As a result of this exemplary arrangement, only two components - 1) the sample collection device (100) containing the swab device (200); and 2) the analytical assay device (600) - are contaminated and are disposed using biohazard disposal procedures.

FIG. 2A is a drawing of an exemplary extraction tube body (110)). The extraction tube body (110) comprises a top end (101), a central portion (107), and a bottom end (102). The top end (101) comprises top end threads (104). The bottom end (102) comprises bottom end threads (108) and a dropper feature (103). In some embodiments, the extraction tube body (110) further comprises an optional lip (105) and support flange (106). In some embodiments, the extraction tube body (110) comprises a fill line (111), e.g., marking a volume of approximately and/or at least 300 µL.

FIG. 2B is a drawing of an exemplary extraction tube body (110)). The extraction tube body (110) comprises a top end (101), a central portion (107), and a bottom end (102). The bottom end (102) comprises a dropper feature (103). In some embodiments, the extraction tube body (110) further comprises an optional lip (105) and support flange (106).

FIG. 3A is a drawing of a top cap (120) shown in a top view. In some embodiments, the top cap (120) comprises a plurality of ridges (121) on the periphery.

FIG. 3B is a drawing of a top cap (120) shown in a side view. In some embodiments, the top cap (120) comprises a plurality of ridges (121) on the periphery.

FIG. 3C is a drawing of a top cap (120) shown in an oblique view. In some embodiments, the top cap (120) comprises a plurality of ridges (121) on the periphery. In some embodiments, the top cap (120) comprises threads (122), e.g., to engage with top end threads of an extraction tube body.

FIG. 4A is a drawing of a bottom cap (130) shown in a top view. In some embodiments, the bottom cap (130) comprises a plurality of ridges (131) on the periphery.

FIG. 4B is a drawing of a bottom cap (130) shown in a side view. In some embodiments, the bottom cap (130) comprises a plurality of ridges (131) on the periphery. In some embodiments, the bottom cap (130) comprises threads (132), e.g., to engage with bottom end threads of an extraction tube body.

FIG. 5A is a drawing of an embodiment of an extraction tube body (110) comprising a separation component (140) and a swab device (200) comprising a swab end (210) and a swab handle (220) comprising a breakable junction (230).

FIG. 5B is a drawing showing use of the separation component (140) of the extraction tube body (110) to break a swab device (200) at a breakable junction (230) by pinching (700) the extraction tube body (110) to cause the separation component (140) to interact with the breakable junction (230) to break the swab handle (220) at the breakable junction (230).

It is to be understood that thefigures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein are devices, kits, systems, and methods for collecting biological samples for analysis and for the safe disposal of sample collection devices after their use. The devices, kits, systems, and methods find use, for example, for providing biological samples for testing and for disposing of biohazard materials by users in settings that may not be equipped with the professional biohazard disposal systems of laboratories, hospitals, and medical clinics.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present or “absent”) is used in a relative sense to describe the amount or level of a particular entity (e.g., an analyte). For example, when an analyte is said to be “present” in a test sample, it means the level or amount of this analyte is above a pre-determined threshold; conversely, when an analyte is said to be “absent” in a test sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods.

As used herein, the term “configured” refers to a component, module, system, subsystem, etc. that is constructed to carry out the indicated function.

As used herein the term “pathogen” refers to an organism, including a microorganism, which causes disease in another organism (e.g., animals (e.g., humans) and plants) by directly infecting the other organism, or by producing agents that causes disease in another organism (e.g., bacteria that produce pathogenic toxins and the like). As used herein, pathogens include, but are not limited to prokaryotes and eukaryotes (e.g., any member of the Bacteria, Archaea, and/or Eukaryota) and thus the term includes pathogenic organisms described as bacteria, eukaryotes, archaebacteria, protozoa, fungi, nematodes, viroids and viruses, or any combination thereof, wherein a pathogen is capable, either by itself or in concert with another pathogen, of eliciting disease in vertebrates including but not limited to mammals, and including but not limited to humans. As used herein, the term “pathogen” also encompasses microorganisms which may not ordinarily be pathogenic in a non-immunocompromised host. Specific nonlimiting examples of viral pathogens include Herpes simplex virus (HSV)1, HSV2, Epstein Barr virus (EBV), cytomegalovirus (CMV), human Herpes virus (HHV) 6, HHV7, HHV8, Varicella zoster virus (VZV), hepatitis C, hepatitis B, adenovirus, Eastern Equine Encephalitis Virus (EEEV), West Nile virus (WNE), JC virus (JCV), BK virus (BKV), MERS, SARS, SARS-CoV-2, influenza virus, Zika virus, Chikungunya virus, Aura virus, Bebaru virus, Cabassou virus, Dengue virus, Fort morgan virus, Getah virus, Kyzylagach virus, Mayoaro virus, Middleburg virus, Mucambo virus, Ndumu virus, Pixuna virus, Tonate virus, Triniti virus, Una virus, Western equine encephalomyelitis virus, Whataroa virus, Sindbis virus (SIN), Semliki forest virus (SFV), Venezuelan equine encephalomyelitis virus (VEE), Ross River virus, human immunodeficiency virus (HIV-1, HIV-2), and HTLV (HTLV-1, HTLV-2, HTLV-3, and HTLV-4). See, e.g., Strauss and Strauss, Microbiol. Rev., 58:491-562 (1994), incorporated herein by reference.

As used herein, the term “microorganism” includes prokaryotic and eukaryotic microbial species from the Domains of Archaea, Bacteria, and Eukarya, the latter including yeast and filamentous fungi, protozoa, algae, or higher Protista. The terms “microbial cells” and “microbes” are used interchangeably with the term microorganism.

The terms “bacteria” and “bacterium” refer to prokaryotic organisms of the domain Bacteria in the three-domain system (see, e.g., Woese CR, et al., Proc Natl Acad Sci U S A 1990, 87: 4576 - 79). It is intended that the terms encompass all microorganisms considered to be bacteria including Mycobacterium, Mycoplasma, Chlamydia, Actinomyces, Streptomyces, and Rickettsia. All forms of bacteria are included within this definition including cocci, bacilli, spirochetes, spheroplasts, protoplasts, etc. In some embodiments, bacteria are capable of causing disease and product degradation or spoilage. Accordingly, “Bacteria”, or “Eubacteria”, refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram-positive bacteria” include cocci, nonsporulating rods, and sporulating rods. The genera of Gram-positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

As used herein, the term “sample” refers to a material to be tested for the presence or amount of an analyte, e.g., a pathogen or a part or component thereof. Preferably, a sample is a fluid sample, preferably a liquid sample, and most preferably a nasopharyngeal sample collected by a swab device as described herein. For example, a sample may be a bodily fluid such as blood, serum, plasma, ocular fluid, urine, mucus, semen, nasopharyngeal swab fluid, throat swab, tears, sweat, or saliva. Viscous liquid, semi-solid, or solid specimens may be used to create liquid solutions, eluates, suspensions, or extracts that can be samples. For example, throat, nasal (e.g., nasopharyngeal swab samples), or genital swabs may be suspended in a liquid solution (e.g., a buffer solution) to make a sample.

As used herein, the term “coupled” refers to two or more components that are secured, by any suitable means (e.g., by engaged threads), together. Accordingly, in some embodiments, the statement that two or more parts or components are “coupled” shall mean that the parts are joined together and are directly in contact with each other unless specifically described in a different configuration. For example, as used herein, “indirectly coupled” means that two elements are in contact with each other through one or more intermediate parts or components. As used herein, “fixedly coupled” or “fixed” means that two components are coupled so as to move as one while maintaining a constant orientation relative to each other. Accordingly, when two elements are coupled, all portions of those elements are coupled. A description, however, of a specific portion of a first element being coupled to a second element, e.g., an axle first end being coupled to a first wheel, means that the specific portion of the first element is disposed closer to the second element than the other portions thereof. Further, an object resting on another object held in place only by gravity is not “coupled” to the lower object unless the upper object is otherwise maintained substantially in place. That is, for example, a book on a table is not coupled thereto, but a book glued to a table is coupled thereto.

In some embodiments, the systems and methods provided herein relate to the reduction of biohazard (e.g., live virus) risk during the sample preparation process for analytical testing. For example, the emergence of the COVID-19 pandemic has created a need for substantial testing outside of traditional laboratory and hospital environments. Some tests, such as lateral flow-based immunoassays may be readily conducted outside medical facilities (e.g., at home or in medical clinics) by unsophisticated users. The systems and methods provided herein provide new ways to minimize the exposure risk of the users, the community, healthcare workers, and laboratory personnel to dangerous agents such as SARS-CoV-2 (the causative virus for the COVID-19 disease) during sample preparation and testing.

The invention is not limited by the nature of the dangerous materials. In some embodiments, the materials are chemical or biological. In some embodiments, biological materials are from a biological sample such as saliva, mucus, urine, feces, blood or blood components (e.g., plasma, serum), wound compounds, tissue, sweat, lesion components, and the like. In some embodiments, the dangerous agent associated with the biological materials is a pathogen. Pathogens include, but are not limited to, viruses, bacteria, parasites, cells, and the like. In some embodiments, the sample is taken from a human. In some embodiments, the sample is taken from a non-human animal (e.g., a companion animal (e.g., dog, cat, horse, etc.), a livestock animal (e.g., cow, pig, chicken, etc.), or a wild animal (e.g., bird, fish, deer, tick, etc.)).

### Swab Device and Sample Collection Device

Many collection systems employ a swab device. As used herein, a swab device is an absorbent pad or piece of material used for cleaning wounds, applying medication, or taking specimens. In some embodiments, a swab device comprises a swab end comprising a wad of absorbent material wound around one end (e.g., a distal end) of a small stick providing a swab handle. The technology is not limited by the nature of the material used for the swab end. Suitable materials include nylon, rayon, cotton, polyester, polyurethane, and alginate polymer. Such materials may be formed into microstructures including, but not limited to, tightly wound, knitted, flocked fiber, and reticulated structures.

The swab end of the swab device is typically contacted with a sample to collect a portion of the sample. The collected portion is then available for transfer to an analytical assay device and/or analytical system for sample preparation and analytical testing.

Provided herein are devices for capturing or otherwise isolating a sample from a swab device that has contacted a potentially dangerous sample. Several complementary and alternative embodiments are provided herein.

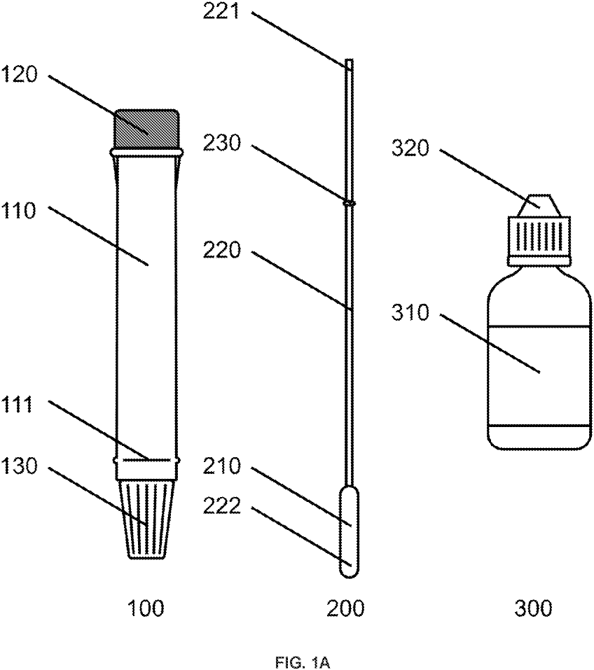

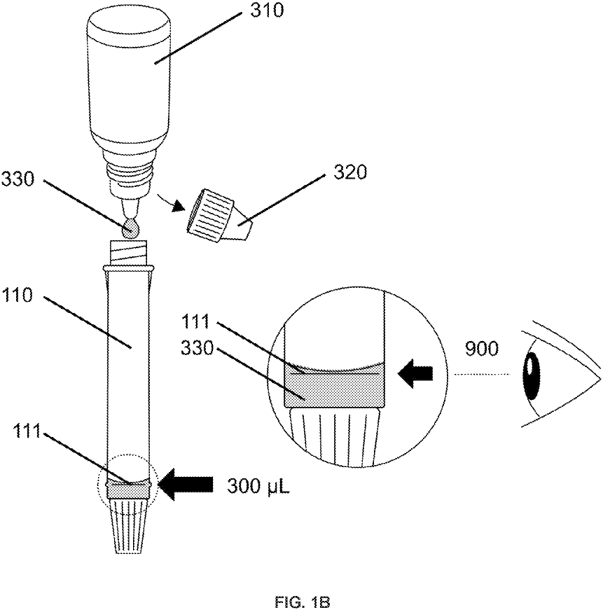

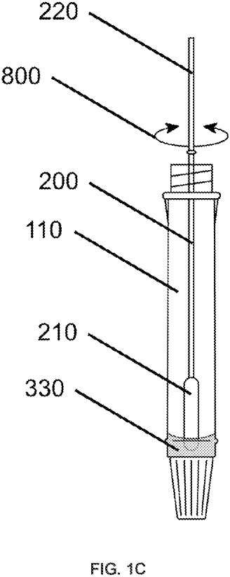

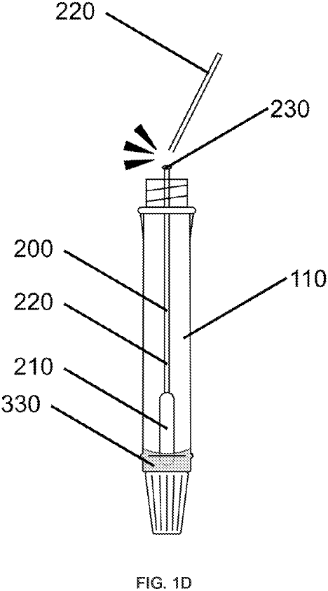

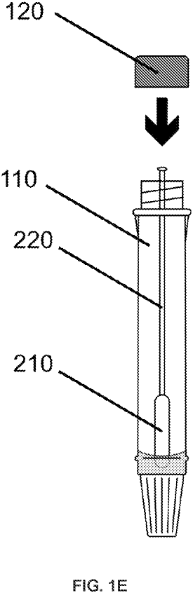

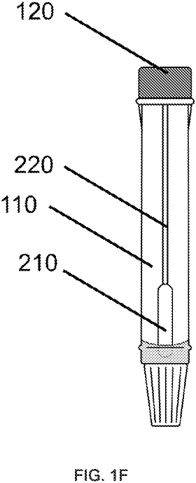

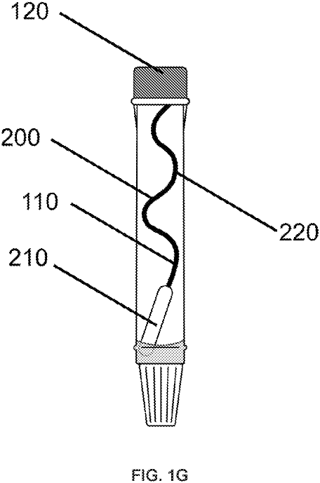

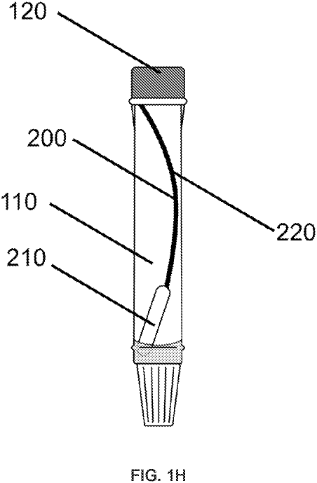

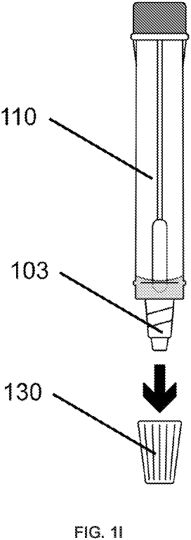

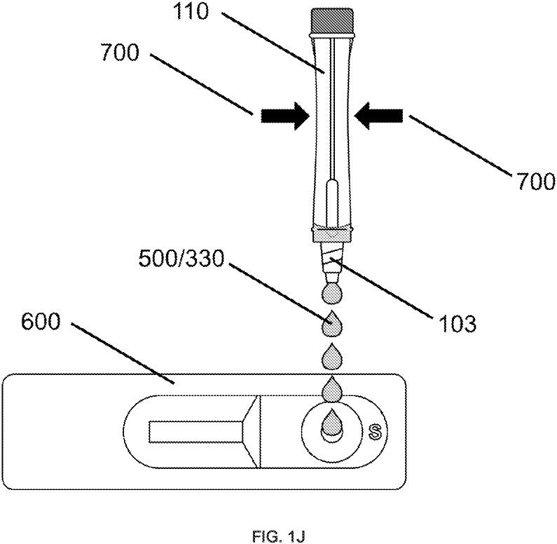

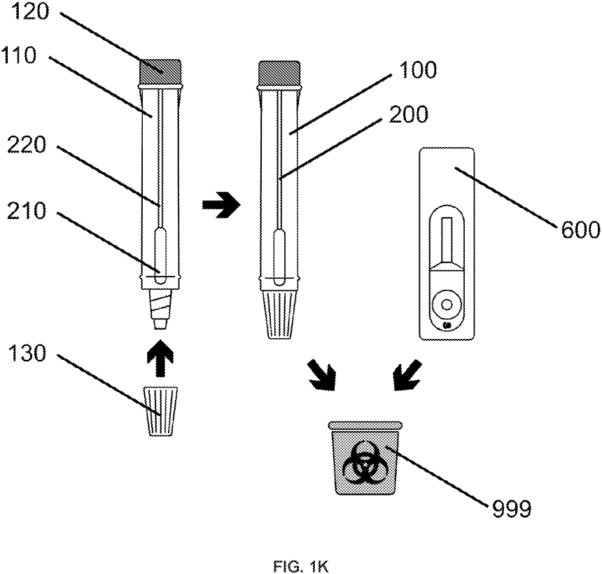

In some embodiments, (e.g., as shown in FIGS. 1A-1K), a swab device (200) is provided with a swab handle (220) where the swab handle (220) is designed to be held by a user (e.g., a user hand) at its proximal end (221) during sample collection. The distal end (222) of the swab handle (220) comprises a swab end (210). In some embodiments, the swab handle (220) comprises a breakable junction (230) between the proximal end (221) of the swab handle (220) and the swab end (210). In some embodiments, the breakable junction (130) is between approximately 5 to 9 cm from the swab end (e.g., approximately 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 cm from the swab end). In some embodiments, the breakable junction (230) is a notched, serrated, scored, or otherwise structurally weakened portion of the swab handle (220) that is readily torn or broken to separate the swab end (210) from at least a portion of the swab handle (220) after the swab end (210) collects a biological sample and is inserted into an extraction tube body (110). In some embodiments, the swab handle (220) is made of a flexible material, e.g., a plastic such as polyethylene, polypropylene, polyester, polyvinyl chloride, acrylonitrile-butadiene-styrene, and polyamide (e.g., NYLON). In some such embodiments, the flexible material of the swab handle (220) facilitates bending of the swab handle (220) and placing some or all of the swab device (200), including the swab handle (220), into an extraction tube body (110) (e.g., even without breaking the swab handle (220) at the breakable junction (230) to separate the swab end (210) from at least a portion of the swab handle (220), although both approaches may be used together).

In some embodiments, (e.g., as shown in FIGS. 1A-1K), the technology further provides a sample collection device (100) comprising an extraction tube body (110) that receives all or a portion of the swab device (200) and that securely and safely isolates the swab end (210) containing the biological sample and prevents exposure of users to potentially dangerous and/or biohazard materials contained in the biological sample. Following sample collection, the swab end (210) and any components associated with the swab end (210), e.g., at least a portion of the swab handle (220), are placed into and stored in the extraction tube body (110) by sealing the extraction tube body (110) with a top cap (120) and a bottom cap (130). The extraction tube body (110) comprises a fill line (111) that serves a reference for filling the extraction tube body (110) with a buffer solution (e.g., provided by a dropper bottle (300)). In some embodiments, the extraction tube body (110) has a sufficient length above the fill line (111) to prevent splash of buffer solution (330) from reaching the top end (101) of the extraction tube body (110) when buffer solution (330) is added to the extraction tube body (110).

Furthermore, as described herein, the technology provides a number of features that minimize the number of items that require disposal and/or that minimize the potential for dangerous material to be transferred outside the collection component (e.g., to contact a user and/or the environment).

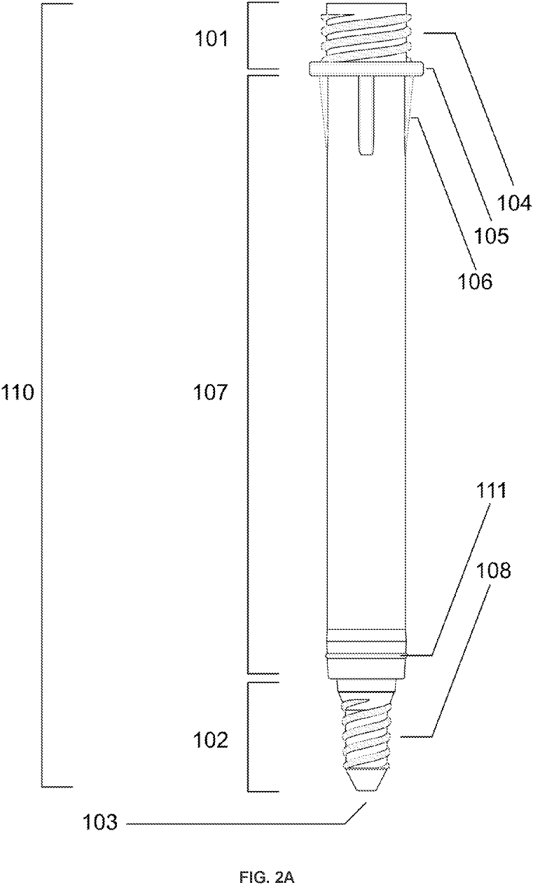

An exemplary collection component is shown in FIGS. 2A and 2B. As shown in FIG. 2A, in some embodiments, the sample collection device comprises an extraction tube body (110). In some embodiments, the extraction tube body (110) comprises a top end (101), a central portion (107), and a bottom end (102). In some embodiments, the extraction tube body (110) has a length that is sufficiently long and narrow to minimize and/or eliminate splash-over of sample (e.g., biohazard materials) outside of the extraction tube body (110).

In some embodiments, the bottom end (102) comprises a dropper feature (103). In some embodiments, the dropper feature (103) is cylindrical. In some embodiments, the dropper feature (103) is generally cone shaped and/or comprises a frustum of a cone. In some embodiments, the dropper feature (103) comprises a portion that is cylindrical and a portion that is generally cone shaped and/or comprises a frustum of a cone. In some embodiments, the dropper feature (103) comprises a number of portion(s) that is/are cylindrical and a number of portion(s) that is/are generally cone shaped and/or that comprise(s) a frustum of a cone. In some embodiments, the dropper feature (103) comprises an angled tip. In some embodiments, the dropper feature (103) has the same or similar diameter as the extraction tube body (110). In some embodiments, the dropper feature (103) has a diameter that is smaller than the diameter of the extraction tube body (110). In some embodiments, the extraction tube body (110) further comprises an optional lip (105) and support flange (106). In some embodiments, the support flange (106) provides supplementary rigidity and/or support to the top end (101) of the extraction tube body (110). In some embodiments, the lip (105) provides a stop for the top cap (120), e.g., to stop motion of the top cap (120) when coupled to the top end (101) of the extraction tube body (110). In some embodiments, the lip (105) provides supplementary rigidity and/or support to the top end (101) of the extraction tube body (110), e.g., to maintain the cross-sectional round shape of the top end (101) of the extraction tube body (110) and thus maximize the area of the opening of the top end (101) of the extraction tube body (110) into which a swab device 200 may be inserted.

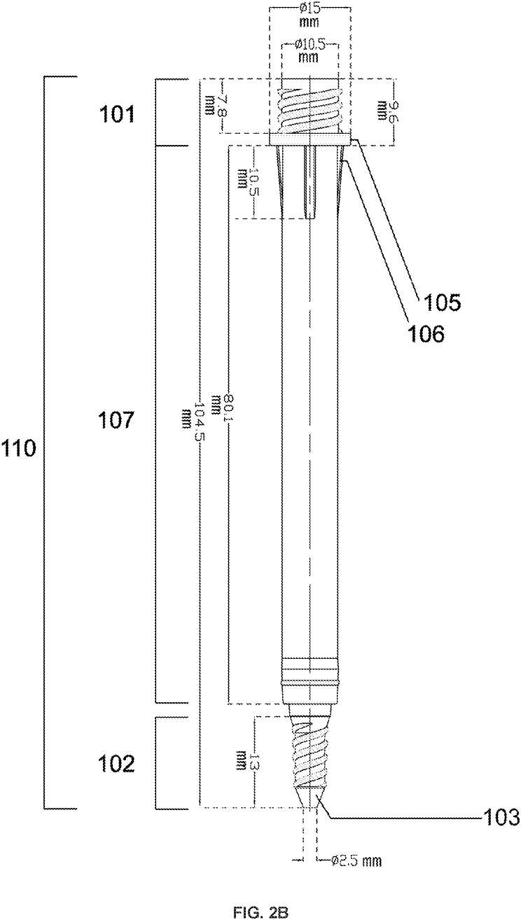

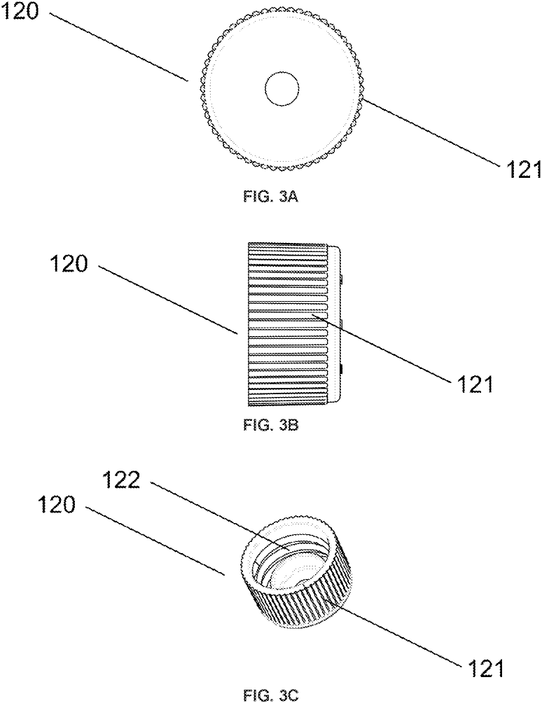

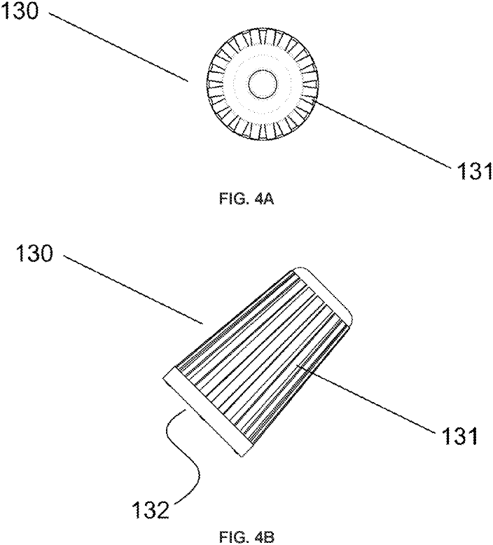

In some embodiments, the top end (101) comprises a set of top end threads (104). In some embodiments, the bottom end (102) comprises a set of bottom end threads (108). In some embodiments, the top end (101) is configured to accept a top cap (120) (e.g., as shown in FIGS. 3A, 3B, and 3C), e.g., the top end threads (104) are configured to engage with a set of threads (122) of the top cap (120). In some embodiments, the bottom end (102) is configured to accept a bottom cap (130) (e.g., as shown in FIGS. 4A and 4B), e.g., the bottom end threads (108) are configured to engage with a set of threads (132) of the bottom cap (130). Accordingly, embodiments provide that the top cap (120) is coupled to the top end (101) and/or the bottom cap (130) is coupled to the bottom end (102).

However, the technology is not limited in the mode of attachment that finds use in coupling the top cap (120) to the top end (101) and/or that finds use in coupling the bottom cap (130) to the bottom end (102). In the illustrated embodiment, the top cap (120) is coupled to the top end (101) by a threaded connection. In some embodiments, the top cap (120) and the top end (101) are coupled together by another suitable mechanical connection type (e.g., a snap-on connection, a press fit connection, a barbed connection, a bayonet-style connection, a tab and slot connection, etc.) As such, the top cap (120) is secured to the top end (101) by a mechanical connection. In the illustrated embodiment, the top cap (120) is secured to and released from the top end (101) with the threaded connection without the use of tools (e.g., the coupling is a tool-free connection).

In the illustrated embodiment, the bottom cap (130) is coupled to the bottom end (102) by a threaded connection. In some embodiments, the bottom cap (130) and the bottom end (102) are coupled together by another suitable mechanical connection type (e.g., a snap-on connection, a press fit connection, a barbed connection, a bayonet-style connection, a tab and slot connection, etc.) As such, the bottom cap (130) is secured to the bottom end (102) by a mechanical connection. In the illustrated embodiment, the bottom cap (130) is secured to and released from the bottom end (102) with the threaded connection without the use of tools (e.g., the coupling is a tool-free connection).

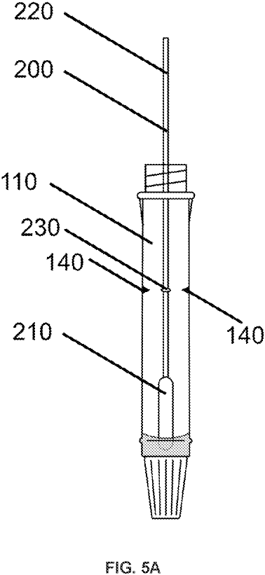

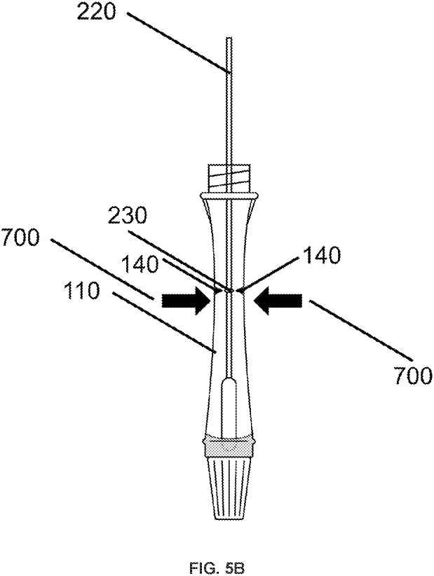

An exemplary top cap (120) is shown in FIGS. 5A and 5B. in some embodiments, the top cap (120) comprises a plurality of ridges (121) on its periphery. In some embodiments, the ridges (121) increase friction between user fingers and the top cap (120) and thus facilitate gripping and manipulating (e.g., rotating) the top cap (120) by a user.

In some embodiments, a user manipulates (e.g., rotates) the top cap (120) to place the top cap (120) on the extraction tube body (110), e.g., by engaging the threads (122) of the top cap (120) with the top end threads (104) and rotating the top cap (120) with respect to the extraction tube body (110) to couple the top cap (120) with the extraction tube body (110). In some embodiments, a user manipulates (e.g., rotates) the top cap (120) to remove the top cap (120) from the extraction tube (100), e.g., by rotating the top cap (120) with respect to the extraction tube body (110) to disengage the threads (122) of the top cap (120) from the top end threads (104) and to decouple the top cap (120) from the extraction tube body (110). In some embodiments, the top cap (120) comprises a plurality of ridges (121) that increase friction between user fingers and the top cap (120) and thus facilitate gripping and manipulating (e.g., rotating) the top cap (120) by a user.

In some embodiments, a user manipulates (e.g., rotates) the bottom cap (130) to place the bottom cap (130) on the extraction tube body (110), e.g., by engaging the threads (132) of the bottom cap (130) with the bottom end threads (108) and rotating the bottom cap (130) with respect to the extraction tube body (110) to couple the bottom cap (130) with the extraction tube body (110). In some embodiments, a user manipulates (e.g., rotates) the bottom cap (130) to remove the bottom cap (130) from the extraction tube body (110), e.g., by rotating the bottom cap (130) with respect to the extraction tube body (110) to disengage the threads (132) of the bottom cap (130) from the bottom end threads (108) and to decouple the bottom cap (130) from the extraction tube body (110). In some embodiments, the bottom cap (130) comprises a plurality of ridges (131) that increase friction between user fingers and the bottom cap (130) and thus facilitate gripping and manipulating (e.g., rotating) the bottom cap (130) by a user.

In some embodiments, the swab device (200) has a length that is longer than a length that is the length of the central portion (107) of the extraction tube body (110) plus the length of the top end (101) of the extraction tube body (110), e.g., so that a portion of the swab device (200) (e.g., a portion of the swab handle (220)) extends from the top of the extraction tube body (110) when the swab device (200) is inserted into the extraction tube body (110) and the swab end (210) of the swab device (200) is near the bottom end (102) of the extraction tube body (110). See, e.g., FIG. 1C.

For example, e.g., as show in FIG. 1A, the swab device (200) has a length defined by the swab end (210) and the swab handle (220). When the swab device (200) is in an unbroken state and is placed in the extraction tube body (110) with the swab end (210) near the bottom end (102) of the extraction tube body (110), the proximal end (221) of the swab handle (220) extends from the top end (101) of the extraction tube body (110). See, e.g., FIG. 1C. As shown in FIG. 1A, the swab handle (220) of the swab device (200) comprises a breakable junction (230). In some embodiments, the breakable junction (230) is a notched, serrated, scored, or otherwise structurally weakened portion of the swab handle (220) that is readily torn or broken to separate the swab end (210) from at least a portion of the swab handle (220). After breaking the swab handle (220) of the swab device (200) at the breakable junction (230) (e.g., as shown in FIG. 1D), the swab device (200) comprises the swab end (210) and, in some embodiments, at least a portion of the swab handle (220). The broken swab device (200) has a length that fits completely, essentially completely, and/or substantially completely inside the extraction tube body (110) (e.g., as shown in FIG. 1E). Accordingly, the top cap (120) can be placed on the extraction tube body (110) (e.g., as shown in FIG. 1F), e.g., by coupling the top cap (120) to the top end (101) of the extraction tube body (110) (e.g., by engaging the threads (122) of the top cap (120) with the bottom end threads (108)).

Accordingly, in some embodiments, e.g., as shown in FIG. 2A and FIG. 2B, the combined length of the central portion (107) and top portion (101) of the extraction tube body (110) has a length that is approximately 87.9 mm (e.g., approximately 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, 80.0, 80.1, 80.2, 80.3, 80.4, 80.5, 80.6, 80.7, 80.8, 80.9, 81.0, 81.1, 81.2, 81.3, 81.4, 81.5, 81.6, 81.7, 81.8, 81.9, 82.0, 82.1, 82.2, 82.3, 82.4, 82.5, 82.6, 82.7, 82.8, 82.9, 83.0, 83.1, 83.2, 83.3, 83.4, 83.5, 83.6, 83.7, 83.8, 83.9, 84.0, 84.1, 84.2, 84.3, 84.4, 84.5, 84.6, 84.7, 84.8, 84.9, 85.0, 85.1, 85.2, 85.3, 85.4, 85.5, 85.6, 85.7, 85.8, 85.9, 86.0, 86.1, 86.2, 86.3, 86.4, 86.5, 86.6, 86.7, 86.8, 86.9, 87.0, 87.1, 87.2, 87.3, 87.4, 87.5, 87.6, 87.7, 87.8, 87.9, 88.0, 88.1, 88.2, 88.3, 88.4, 88.5, 88.6, 88.7, 88.8, 88.9, 89.0, 89.1, 89.2, 89.3, 89.4, 89.5, 89.6, 89.7, 89.8, 89.9, 90.0, 90.1, 90.2, 90.3, 90.4, 90.5, 90.6, 90.7, 90.8, 90.9, 91.0, 91.1, 91.2, 91.3, 91.4, 91.5, 91.6, 91.7, 91.8, 91.9, 92.0, 92.1, 92.2, 92.3, 92.4, 92.5, 92.6, 92.7, 92.8, 92.9, 93.0, 93.1, 93.2, 93.3, 93.4, 93.5, 93.6, 93.7, 93.8, 93.9, 94.0, 94.1, 94.2, 94.3, 94.4, 94.5, 94.6, 94.7, 94.8, 94.9, 95.0, 95.1, 95.2, 95.3, 95.4, 95.5, 95.6, 95.7, 95.8, 95.9, 96.0, 96.1, 96.2, 96.3, 96.4, 96.5, 96.6, 96.7, 96.8, 96.9, 97.0, 97.1, 97.2, 97.3, 97.4, 97.5, 97.6, 97.7, 97.8, 97.9, or 98.0 mm).

Thus, in some embodiments, the swab device has a length that is greater than approximately 87.9 mm (e.g., greater than approximately 78.0, 78.1, 78.2, 78.3, 78.4, 78.5, 78.6, 78.7, 78.8, 78.9, 79.0, 79.1, 79.2, 79.3, 79.4, 79.5, 79.6, 79.7, 79.8, 79.9, 80.0, 80.1, 80.2, 80.3, 80.4, 80.5, 80.6, 80.7, 80.8, 80.9, 81.0, 81.1, 81.2, 81.3, 81.4, 81.5, 81.6, 81.7, 81.8, 81.9, 82.0, 82.1, 82.2, 82.3, 82.4, 82.5, 82.6, 82.7, 82.8, 82.9, 83.0, 83.1, 83.2, 83.3, 83.4, 83.5, 83.6, 83.7, 83.8, 83.9, 84.0, 84.1, 84.2, 84.3, 84.4, 84.5, 84.6, 84.7, 84.8, 84.9, 85.0, 85.1, 85.2, 85.3, 85.4, 85.5, 85.6, 85.7, 85.8, 85.9, 86.0, 86.1, 86.2, 86.3, 86.4, 86.5, 86.6, 86.7, 86.8, 86.9, 87.0, 87.1, 87.2, 87.3, 87.4, 87.5, 87.6, 87.7, 87.8, 87.9, 88.0, 88.1, 88.2, 88.3, 88.4, 88.5, 88.6, 88.7, 88.8, 88.9, 89.0, 89.1, 89.2, 89.3, 89.4, 89.5, 89.6, 89.7, 89.8, 89.9, 90.0, 90.1, 90.2, 90.3, 90.4, 90.5, 90.6, 90.7, 90.8, 90.9, 91.0, 91.1, 91.2, 91.3, 91.4, 91.5, 91.6, 91.7, 91.8, 91.9, 92.0, 92.1, 92.2, 92.3, 92.4, 92.5, 92.6, 92.7, 92.8, 92.9, 93.0, 93.1, 93.2, 93.3, 93.4, 93.5, 93.6, 93.7, 93.8, 93.9, 94.0, 94.1, 94.2, 94.3, 94.4, 94.5, 94.6, 94.7, 94.8, 94.9, 95.0, 95.1, 95.2, 95.3, 95.4, 95.5, 95.6, 95.7, 95.8, 95.9, 96.0, 96.1, 96.2, 96.3, 96.4, 96.5, 96.6, 96.7, 96.8, 96.9, 97.0, 97.1, 97.2, 97.3, 97.4, 97.5, 97.6, 97.7, 97.8, 97.9, or 98.0 mm).

In some embodiments, the swab device and/or swab handle has a length that is approximately 70 to 100 mm (e.g., 70.0, 70.1, 70.2, 70.3, 70.4, 70.5, 70.6, 70.7, 70.8, 70.9, 71.0, 71.2, 71.4, 71.6, 71.8, 72.0, 72.2, 72.4, 72.6, 72.8, 73.0, 73.2, 73.4, 73.6, 73.8, 74.0, 74.2, 74.4, 74.6, 74.8, 75.0, 75.2, 75.4, 75.6, 75.8, 76.0, 76.2, 76.4, 76.6, 76.8, 77.0, 77.2, 77.4, 77.6, 77.8, 78.0, 78.2, 78.4, 78.6, 78.8, 79.0, 79.2, 79.4, 79.6, 79.8, 80.0, 80.2, 80.4, 80.6, 80.8, 81.0, 81.2, 81.4, 81.6, 81.8, 82.0, 82.2, 82.4, 82.6, 82.8, 83.0, 83.2, 83.4, 83.6, 83.8, 84.0, 84.2, 84.4, 84.6, 84.8, 85.0, 85.2, 85.4, 85.6, 85.8, 86.0, 86.2, 86.4, 86.6, 86.8, 87.0, 87.2, 87.4, 87.6, 87.8, 88.0, 88.2, 88.4, 88.6, 88.8, 89.0, 89.2, 89.4, 89.6, 89.8, 90.0, 90.2, 90.4, 90.6, 90.8, 91.0, 91.2, 91.4, 91.6, 91.8, 92.0, 92.2, 92.4, 92.6, 92.8, 93.0, 93.2, 93.4, 93.6, 93.8, 94.0, 94.2, 94.4, 94.6, 94.8, 95.0, 95.2, 95.4, 95.6, 95.8, 96.0, 96.2, 96.4, 96.6, 96.8, 97.0, 97.2, 97.4, 97.6, 97.8, 98.0, 98.2, 98.4, 98.6, 98.8, 99.0, 99.2, 99.4, 99.6, 99.8, or 100.0 mm).

In some embodiments, e.g., as shown in FIG. 4B, the top end (101) has a length that is approximately 7.8 mm (e.g., approximately 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0 mm) (e.g., in embodiments excluding the optional lip) or approximately 9.6 mm (e.g., approximately 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0 mm) (e.g., in embodiments including the optional lip). In some embodiments, e.g., as shown in FIG. 4B, the central portion (107) of extraction tube body (110) has an outer diameter that is approximately 10.5 mm (e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0 mm). In some embodiments, the extraction tube body (110) (e.g., the top end (101) and/or the central portion (107) of the extraction tube body (110)) has an inner diameter that is approximately 9 mm (e.g., 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mm).

In some embodiments, e.g., as shown in FIG. 4B, the bottom end (102) has a length that is approximately 13 mm (e.g., approximately 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, or 16.0 mm). In some embodiments, e.g., as shown in FIG. 4B, the dropper feature (103) has an outer diameter that is approximately 2.5 mm (e.g., approximately 1.5, 1.6, 1.7, 1.8, 1.9, 2.0, 2.1, 2.2, 2.3, 2.4, or 2.5 mm). In some embodiments, the dropper feature (103) has an inner diameter that is approximately 1.6 mm (e.g., approximately 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or 2.00 mm). In some embodiments, the dropper feature (103) provides (e.g., outputs) 1-10 (e.g., 1, 2, 3, 4, 5, 6, 7, 8, 9, or 10) drops of a sample. In some embodiments, a drop of a sample has a volume of approximately 50 µl (e.g., approximately 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.4, 46.5, 46.6, 46.7, 46.8, 46.9, 47.0, 47.1, 47.2, 47.3, 47.4, 47.5, 47.6, 47.7, 47.8, 47.9, 48.0, 48.1, 48.2, 48.3, 48.4, 48.5, 48.6, 48.7, 48.8, 48.9, 49.0, 49.1, 49.2, 49.3, 49.4, 49.5, 49.6, 49.7, 49.8, 49.9, 50.0, 50.1, 50.2, 50.3, 50.4, 50.5, 50.6, 50.7, 50.8, 50.9, 51.0, 51.1, 51.2, 51.3, 51.4, 51.5, 51.6, 51.7, 51.8, 51.9, 52.0, 52.1, 52.2, 52.3, 52.4, 52.5, 52.6, 52.7, 52.8, 52.9, 53.0, 53.1, 53.2, 53.3, 53.4, 53.5, 53.6, 53.7, 53.8, 53.9, 54.0, 54.1, 54.2, 54.3, 54.4, 54.5, 54.6, 54.7, 54.8, 54.9, 55.0, 55.1, 55.2, 55.3, 55.4, 55.5, 55.6, 55.7, 55.8, 55.9, 56.0, 56.1, 56.2, 56.3, 56.4, 56.5, 56.6, 56.7, 56.8, 56.9, 57.0, 57.1, 57.2, 57.3, 57.4, 57.5, 57.6, 57.7, 57.8, 57.9, 58.0, 58.1, 58.2, 58.3, 58.4, 58.5, 58.6, 58.7, 58.8, 58.9, 59.0, 59.1, 59.2, 59.3, 59.4, 59.5, 59.6, 59.7, 59.8, 59.9, or 60.0 µl).

The technology is not limited in the material from which the extraction tube body is made. For example, in some embodiments, the extraction tube body is made from a polymer that is bendable and/or flexible. Examples of flexible polymers include, e.g., polyethylene (e.g., high density polyethylene (HDPE), low density polyethylene (LDPE)), polypropylene, polyamide, and similar materials.

As shown in FIG. 5A, in some embodiments, the extraction tube body (110) comprises a separation component (140) that facilitates separation of the swab end (210) from at least a portion of the swab handle (220). For example, in some embodiments, the separation component (140) comprises a cutting edge or cutting ridge that facilitates separation of the swab end (210) from at least a portion of the swab handle (220) after the swab device (200) has been inserted into the extraction tube body (110). In some embodiments, the separation component (140) is positioned to interact with the breakable junction (230) to facilitate breaking the swab handle (220) at the breakable junction (230). In some embodiments, the breakable junction (230) is a notched, serrated, scored, or otherwise structurally weakened portion of the swab handle (220) that is readily torn or broken (e.g., by interacting with the separation component (140)) to separate the swab end (210) from at least a portion of the swab handle (220). As shown in FIG. 5B, in some embodiments, a user pinches (700) the extraction tube body (110) to cause the separation component (140) to interact with the breakable junction (230) to break the swab handle (220) at the breakable junction (230).

### Kits

In some embodiments, a kit is provided containing components useful, necessary, or sufficient for sample collection and/or sample processing and analysis. In some embodiments, kits comprise one or more bottles or other containers containing a buffer solution, etc. (e.g., storage buffers that stabilize the sample for shipment and/or storage). In some embodiments, e.g., as shown in FIGS. 1A-1K, the technology provides a kit comprising a swab device (200) and a collection device (100). In some embodiments, the extraction tube body (110) of the collection device (100) comprises a buffer solution, e.g., in some embodiments of kits the extraction tube body (110) of the collection device (100) is pre-filled with a buffer solution (330). In some embodiments, the technology provides a kit comprising a swab device (200), a collection device (100), and a dropper bottle (300) comprising a buffer solution (330). In some embodiments, a user of the kit adds buffer solution (330) to the extraction tube body (110) of the collection device (100). The technology is not limited in how the user adds buffer solution (330) to the extraction tube body (110) of the collection device (100). While an exemplary embodiment provides a dropper bottle (300) comprising a buffer solution (330), embodiments also provide that buffer solution (330) is added to the extraction tube body (110) of the collection device (100) by a user using a pipette, by pouring, from a vial, etc. In some embodiments, buffer solution (330) is provided as a pre-measured amount and a user adds the entire volume of buffer solution (330) to the extraction tube body (110) of the collection device (100). In some embodiments, buffer solution (330) is provided in a volume that is greater than the amount added to the extraction tube body (110) of the collection device (100) and a user measures an appropriate amount of buffer solution (330) to be added to the extraction tube body (110) of the collection device (100). In some embodiments, a user adds buffer solution (330) to the extraction tube body (110) of the collection device (100) until the buffer solution (330) reaches a fill line (111) on the extraction tube body (110) of the collection device.

In some embodiments, the technology provides a kit comprising: a) a swab device (200) comprising a swab end (210) and a swab handle (220); b) a sample collection device (100) comprising an extraction tube body (110) comprising a dropper feature (103); and c) a buffer solution (330) (e.g., a dropper bottle (300) comprising a dropper bottle body (310) comprising buffer solution (330); and a dropper bottle cap (320)). In some embodiments, the sample collection device (100) of the kit comprises a top cap (120) and a bottom cap (130). In some embodiments, the extraction tube body (110) comprises a top end (101) configured to accept the top cap (120); and the extraction tube body (110) comprises a bottom end (102) configured to accept the bottom cap (130). In some embodiments, the swab device (200) of the kit comprises a breakable junction (230). In some embodiments, the swab handle (220) of the swab device (200) comprises the breakable junction (230). In some embodiments, the swab handle (220) of the swab device (200) comprises a proximal end (221) and a distal end (222), the swab handle (220) comprises the swab end (210) at the distal end (222), and the swab handle (220) comprises the breakable junction (230) between the proximal end (221) of the swab handle (220) and the distal end (222) of the swab handle (220). In some embodiments, kits further comprise an analytical assay device (600). In some embodiments, kits further comprise a biohazard disposal container (999). In some embodiments, the extraction tube body (110) of the collection device (100) of the kit comprises a fill line (111). As shown in FIGS. 5A and 5B, in some embodiments, the extraction tube body (110) comprises a separation component (140) that interacts with the breakable junction (230) to facilitate breaking the swab handle (220) at the breakable junction (230).

### Systems

In some embodiments, a system is provided containing components useful, necessary, or sufficient for sample collection and/or sample processing and analysis. In some embodiments, systems comprise one or more bottles or other containers containing a buffer solution, etc. (e.g., storage buffers that stabilize the sample for shipment and/or storage). In some embodiments, e.g., as shown in FIGS. 1A-1K, the technology provides a system comprising a swab device (200) and a collection device (100). In some embodiments, the extraction tube body (110) of the collection device (100) comprises a buffer solution, e.g., in some embodiments of systems the extraction tube body (110) of the collection device (100) is pre-filled with a buffer solution (330). In some embodiments, the technology provides a system comprising a swab device (200), a collection device (100), and a dropper bottle (300) comprising a buffer solution (330). In some embodiments, a user of the system adds buffer solution (330) to the extraction tube body (110) of the collection device (100). The technology is not limited in how the user adds buffer solution (330) to the extraction tube body (110) of the collection device (100). While an exemplary embodiment provides a dropper bottle (300) comprising a buffer solution (330), embodiments also provide that buffer solution (330) is added to the extraction tube body (110) of the collection device (100) by a user using a pipette, by pouring, from a vial, etc. In some embodiments, buffer solution (330) is provided as a pre-measured amount and a user adds the entire volume of buffer solution (330) to the extraction tube body (110) of the collection device (100). In some embodiments, buffer solution (330) is provided in a volume that is greater than the amount added to the extraction tube body (110) of the collection device (100) and a user measures an appropriate amount of buffer solution (330) to be added to the extraction tube body (110) of the collection device (100). In some embodiments, a user adds buffer solution (330) to the extraction tube body (110) of the collection device (100) until the buffer solution (330) reaches a fill line (111) on the extraction tube body (110) of the collection device.

In some embodiments, the technology provides a system comprising: a) a swab device (200) comprising a swab end (210) and a swab handle (220); b) a sample collection device (100) comprising an extraction tube body (110) comprising a dropper feature (103); and c) a buffer solution (330) (e.g., a dropper bottle (300) comprising a dropper bottle body (310) comprising buffer solution (330); and a dropper bottle cap (320)). In some embodiments, the sample collection device (100) of the system comprises a top cap (120) and a bottom cap (130). In some embodiments, the extraction tube body (110) comprises a top end (101) configured to accept the top cap (120); and the extraction tube body (110) comprises a bottom end (102) configured to accept the bottom cap (130). In some embodiments, the swab device (200) of the system comprises a breakable junction (230). In some embodiments, the swab handle (220) of the swab device (200) comprises the breakable junction (230). In some embodiments, the swab handle (220) of the swab device (200) comprises a proximal end (221) and a distal end (222), the swab handle (220) comprises the swab end (210) at the distal end (222), and the swab handle (220) comprises the breakable junction (230) between the proximal end (221) of the swab handle (220) and the distal end (222) of the swab handle (220). In some embodiments, systems further comprise an analytical assay device (600). In some embodiments, systems further comprise a reader apparatus to read a test result provided by an analytical assay device (600). In some embodiments, systems further comprise a biohazard disposal container (999). In some embodiments, the extraction tube body (110) of the collection device (100) of the system comprises a fill line (111). As shown in FIGS. 5A and 5B, in some embodiments, the extraction tube body (110) comprises a separation component (140) that interacts with the breakable junction (230) to facilitate breaking the swab handle (220) at the breakable junction (230).

### Methods of Using a Swab Device a Sample Collection Device

In some embodiments, methods comprise providing a swab device and a collection device. In some embodiments, methods comprise providing a swab device, a collection device, and a dropper bottle comprising a buffer solution (see, e.g., FIG. 1A). In some embodiments, methods comprise providing a kit or system comprising a swab device and a collection device. In some embodiments, methods comprise providing a kit or system comprising a swab device, a collection device, and a dropper bottle comprising a buffer solution.

In some embodiments, methods comprise adding a buffer solution or extraction solution (e.g., approximately 300 µL (e.g., 250, 260, 270, 280, 290, 300, 310, 320, 330, 340, or 350 µL)) to the sample collection device (e.g., the extraction tube body) (see, e.g., FIG. 1B). In some embodiments, methods comprise observing the buffer solution above a fill line provided on the extraction tube body and/or confirming that a volume of approximately 300 µL has been added to the extraction tube body, e.g., as shown in FIG. 1B. In some embodiments, methods comprise acquiring a sample on a swab device (e.g., acquiring a sample on a swab end of a swab device) comprising a swab handle, placing the swab device into a sample collection device (e.g., an extraction tube body) (see, e.g., FIG. 1C), and, optionally, moving the swab device to facilitate transferring at least a portion of the analyte in the sample from the swab end into a buffer solution in the extraction tube (e.g., by rotating the swab handle approximately 5 times (e.g., 1, 2, 3, 4, 5, 6, 7, 8, 9, or 10 times)) (see, e.g., FIG. 1C). Next, in some embodiments, methods comprise removing the swab handle or a portion thereof (e.g., by breaking the swab handle at a breakable junction) from the swab device (see, e.g., FIG. 1D). In some embodiments, removing the swab handle from the swab device comprises pinching the extraction tube body and/or breaking the swab handle while the swab handle is inside the extraction tube body (e.g., embodiments provide that a user contacts the outside of the extraction tube body to break the swab handle but the user does not contact the swab handle to break the swab handle). In some embodiments, methods comprise pinching the extraction tube body to cause a separation component to interact with the breakable junction and facilitate removing the swab handle or a portion thereof (e.g., by breaking the swab handle at a breakable junction) from the swab device (see, e.g., FIGS. 5A and 5B). Accordingly, in some embodiments, methods comprise contacting a separation component of the extraction tube body to the breakable junction. In some embodiments, methods further comprise sealing the extraction tube body with a top cap (see, e.g., FIG. 1E and FIG. 1F), e.g., by coupling a top cap with a top end of the extraction tube body. In some embodiments, methods further comprise removing a bottom cap from a dropper feature (e.g., from a bottom end of the extraction tube body) (see, e.g., FIG. 1I). In some embodiments, methods further comprise producing and/or outputting one or more drops (e.g., approximately 5 drops (e.g., 1, 2, 3, 4, 5, 6, 7, 8, 9, or 10 drops)) from the extraction tube body (e.g., from the dropper feature) (see, e.g., FIG. 1J). In some embodiments, producing and/or outputting one or more drops from the extraction tube body comprises squeezing the extraction tube body (see, e.g., FIG. 1J) to increase the pressure inside the extraction tube body and force drops through the dropper feature. In some embodiments, drops are provided onto a sample well of an analytical assay device (see, e.g., FIG. 1J), e.g., a lateral flow assay device as described in Int′l Pat. App. No. PCT/US21/026183, which is incorporated herein by reference. In some embodiments, methods comprise observing and/or reading a test result. In some embodiments, observing and/or reading a test result comprises observing one or more visually detectable result lines of a testing cassette. In some embodiments, observing and/or detecting the presence of only a control line (C) and no test line (T) indicates a negative result; and the presence of a test line (T) and a control line (C), regardless of which line appears first, indicates a positive result. In some embodiments, the presence of a test line (T), no matter how faint, indicates a positive result. In some embodiments, if the control line (C) is not visible ]after performing the test, the result is considered invalid. In some embodiments, methods comprise disposing of the collection device and/or analytical assay device (e.g., a lateral flow assay device) (see, e.g., FIG. 1K).

In some embodiments, methods comprise adding a buffer solution or extraction solution (e.g., approximately 300 µL (e.g., 250, 260, 270, 280, 290, 300, 310, 320, 330, 340, or 350 µL)) to the sample collection device (e.g., the extraction tube body) (see, e.g., FIG. 1B). In some embodiments, methods comprise observing the buffer solution above a fill line provided on the extraction tube body and/or confirming that a volume of approximately 300 µL has been added to the extraction tube body, e.g., as shown in FIG. 1B. In some embodiments, methods comprise acquiring a sample on a swab device (e.g., acquiring a sample on a swab end of a swab device) comprising a swab handle, placing the swab device into a sample collection device (e.g., an extraction tube body) (see, e.g., FIG. 1C), and, optionally, moving the swab device to facilitate transferring at least a portion of the analyte in the sample from the swab end into a buffer solution in the extraction tube (e.g., by rotating the swab handle approximately 5 times (e.g., 1, 2, 3, 4, 5, 6, 7, 8, 9, or 10 times)) (see, e.g., FIG. 1C). In some embodiments, methods further comprise sealing the extraction tube body with a top cap (see, e.g., FIG. 1E and FIG. 1F), e.g., by coupling a top cap with a top end of the extraction tube body. In some embodiments, the swab handle is flexible and methods comprise bending and/or compressing the swab handle to seal the extraction tube body with the top cap (see, e.g., FIG. 1G and FIG. 1H). Accordingly, in some embodiments, the swab handle is not broken and is instead bent and/or compressed to seal the swab device comprising the swab handle and swab end inside the extraction tube body. In some embodiments, methods further comprise removing a bottom cap from a dropper feature (e.g., from a bottom end of the extraction tube body) (see, e.g., FIG. 1I). In some embodiments, methods further comprise producing and/or outputting one or more drops (e.g., approximately 5 drops (e.g., 1, 2, 3, 4, 5, 6, 7, 8, 9, or 10 drops)) from the extraction tube body (e.g., from the dropper feature) (see, e.g., FIG. 1J). In some embodiments, producing and/or outputting one or more drops from the extraction tube body comprises squeezing the extraction tube body (see, e.g., FIG. 1J) to increase the pressure inside the extraction tube body and force drops through the dropper feature. In some embodiments, drops are provided onto a sample well of an analytical assay device (see, e.g., FIG. 1J), e.g., a lateral flow assay device as described in Int′l Pat. App. No. PCT/US21/026183, which is incorporated herein by reference. In some embodiments, methods comprise observing and/or reading a test result. In some embodiments, observing and/or reading a test result comprises observing one or more visually detectable result lines of a testing cassette. In some embodiments, observing and/or detecting the presence of only a control line (C) and no test line (T) indicates a negative result; and the presence of a test line (T) and a control line (C), regardless of which line appears first, indicates a positive result. In some embodiments, the presence of a test line (T), no matter how faint, indicates a positive result. In some embodiments, if the control line (C) is not visible ]after performing the test, the result is considered invalid. In some embodiments, methods comprise disposing of the collection device and/or lateral flow assay device (see, e.g., FIG. 1K).

One exemplary embodiment is shown in FIGS. 1A-J. A dropper bottle (300) containing buffer solution is provided with a swab device (200) and a sample collection device (100). Buffer solution (330) is added to the extraction tube body (110) (FIG. 1B). In some embodiments, the extraction tube body (110) comprises a fill line (111) or marking indicating the appropriate volume (e.g., approximately 300 µL (e.g., approximately 250, 260, 270, 280, 290, 300, 310, 320, 330, 340, or 350 µL)) of buffer (330) to add to the extraction tube body (110) (FIG. 1B). The swab device (200) (e.g., the swab end (210) of the swab device (200)) is contacted with a biological sample (e.g., a nasopharyngeal sample). The swab end (210) of the swab device (200) is inserted into the extraction tube body (110) with the swab handle (220) extending out of the extraction tube body (FIG. 1C). In some embodiments, the swab end (210) is squeezed to release at least a portion of the analyte in the sample (e.g., biological materials) into the buffer solution (330). Squeezing can be facilitated by any desired mechanism. In some embodiments, the swab end (210) is compressed against the interior surface of the extraction tube body (110) (e.g., against the interior surface of the extraction tube body (110). In some embodiments, the interior of the extraction tube body (110) is shaped to compress and squeeze the swab end (210), for example, by having a diameter near the bottom end (102) of the extraction tube body (110) that is smaller than the diameter of the unsqueezed swab end (210). In some embodiments, the swab device (200) is swirled or rotated (800) to release at least a portion of the analyte in the sample (e.g., comprising biological materials) into the buffer solution (330).

Next, in some embodiments, at least a portion of the swab handle (220) of the swab device (200) is separated from the swab end (210) (FIG. 1D). This can be accomplished by cutting, breaking, or any desired mechanism. In some embodiments, the swab handle (220) comprises a breakable junction (230) that facilitates cutting, breaking, or any desired mechanism to separate at least a portion of the swab handle (220) of the swab device (200).

In some embodiments, removing at least a portion of the swab handle (220) of the swab device (200) from the swab end (210) comprises pinching the extraction tube body (FIG. 5B) and/or breaking the swab handle (220) while the swab handle (220) is inside the extraction tube body (110) (e.g., embodiments provide that a user contacts the outside of the extraction tube body (110) to break the swab handle (220) but the user does not contact the swab handle (220) to break the swab handle (220)). In some embodiments, methods comprise pinching the extraction tube body (110) to cause a separation component (140) to interact with the breakable junction (130) and facilitate removing the swab handle (220) or a portion thereof (e.g., by breaking the swab handle (220) at a breakable junction (230)) from the swab device (200) (see, e.g., FIGS. 5A and 5B). Accordingly, in some embodiments, methods comprise contacting a separation component (140) of the extraction tube body (110) to the breakable junction (130). A top cap (120) is then secured to the extraction tube body (110), securing the entirety of the swab end (210) (and any remaining portion of the swab handle (220) connected to the swab end (210)) in the interior space of the extraction tube body (110)) (FIG. 1E and FIG. 1F).

In some embodiments, the swab handle (220) is flexible and methods comprise bending and/or compressing the swab handle (220) to seal the extraction tube body (110) with the top cap (120) (FIG. 1G and FIG. 1H). Accordingly, in some embodiments, the swab handle (220) is not broken and is instead bent and/or compressed to seal the swab device (200) comprising the swab handle (220) and swab end (210) completely inside the extraction tube body (110). FIG. 1G and FIG. 1H. Thus, in some embodiments, the top cap (120) is secured to the extraction tube body (110), securing the entirety of the swab device (200) comprising the swab end (210) and the swab handle (220) in the interior space of the extraction tube body (110) (FIG. 1G and FIG. 1H).

In some embodiments, the extraction tube body (110) comprises a dropper feature (103) on its bottom end (102). The dropper feature (103) may be accessed by removing a bottom cap (130), exposing the dropper feature (103) (FIG. 1I). One or more drops of the buffer (330), containing the biological sample (500) (e.g., comprising an analyte), can then be emitted from the extraction tube body (110) (FIG. 1J). In some embodiments, producing drops of the buffer (330) comprising at least a portion of the biological sample (500) (e.g., comprising an analyte) comprises squeezing (700) the exterior surface of the extraction tube body (110) (FIG. 1J). The drops (500/330) can be delivered directly to an analytical assay device (600) (FIG. 1J) for further sample preparation or directly for analytical analysis. For example, the drops (500/330) may be applied to an analytical assay device (600) (e.g., a lateral flow assay device) that provides a yes/no answer about the presence of a particular substance (e.g., an analyte that is, e.g., a pathogen protein, a pathogen nucleic acid, a chemical, a hormone, etc.) in the sample. See, e.g., Int′l Pat. App. No. PCT/US21/026183, which is incorporated herein by reference. The bottom cap (130) can then be reattached to the extraction tube body (110), again securing the swab device (200) (e.g., comprising the swab end (210) and at least a portion of the swab handle (220)) and any unused biological sample within the sample collection device 100 (e.g., within the extraction tube body (110)) (FIG. 1K).

Accordingly, the technology provides methods for disposing of the unused sample (500), swab device (200), and sample collection device (100) safely, e.g., by minimizing and/or eliminating the risk of exposure of a user and/or the environment to a pathogen present in the sample (500). For example, the swab device (200) (and unused sample (500)) are sealed inside the sample collection device (100) and may be discarded. In some embodiments, discarding the swab device (200) (and unused sample (500)) and the sample collection device (100) comprises placing the sample collection device (100) (e.g., comprising the swab device (200) and any unused sample (500) sealed inside the extraction tube body (110) by the top cap (120) and bottom cap (130)) into a biohazard disposal container (999) (e.g., a biohazard disposal bag) for safe disposal according to laboratory safety practices. FIG. 1K. In some embodiments, methods further comprise disposing of an analytical assay device (600). In some embodiments, disposing of the analytical assay device (600) comprises placing the analytical assay device (600) into a biohazard disposal container (999) (e.g., a biohazard disposal bag) for safe disposal according to laboratory safety practices. FIG. 1K. In some embodiments, the sample collection device (100) (e.g., comprising the swab device (200) and any unused sample (500)) and analytical assay device are placed into the same biohazard disposal container (999) (e.g., a biohazard disposal bag). In some embodiments, the sample collection device (100) (e.g., comprising the swab device (200) and any unused sample (500)) and the analytical assay device are each placed into a separate biohazard disposal container (999) (e.g., a biohazard disposal bag) and disposed of separately.

Alternative systems on the market provide some protection against contamination, but do not as thoroughly address each of the criteria of: a) reducing sample preparation exposure risk; b) reducing device exposure risk; and c) reducing accessory disposal risk.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A sample collection device comprising an extraction tube body comprising
a top end, a central portion, and a bottom end, wherein
said top end is configured to accept a top cap;
said bottom end is configured to accept a bottom cap; and
said bottom end comprises a dropper feature.

2. The sample collection device of claim 1 wherein said extraction tube body accepts a swab device.

3. The sample collection device of claim 2 wherein said swab device has a length that is longer than a length that is the length of the central portion plus the length of the top end.

4. The sample collection device of claim 2 wherein said swab device comprises a breakable junction.

5. The sample collection device of claim 4 wherein said extraction tube body comprises a separation component.

6. The sample collection device of claim 5 wherein said separation component is configured to interact with said breakable junction.

7. The sample collection device of claim 1 wherein said extraction tube body comprises a fill line.

8. The sample collection device of claim 7 wherein said fill line marks a volume of approximately 300 µL.

9. The sample collection device of claim 2 wherein said swab device is flexible.

10. The sample collection device of claim 9 wherein said swab device can be bent to fit entirely inside said extraction tube body.

11. The sample collection device of claim 2 wherein said extraction tube body comprises a top cap sealing said top end and a bottom cap sealing said bottom end and at least a part of said swab device is sealed inside said extraction tube body.

12. The sample collection device of claim 2 wherein said extraction tube body comprises a top cap sealing said top end said dropper feature is exposed.

13. The sample collection device of claim 1 wherein said dropper feature outputs droplets of approximately 50 µl.

14. The sample collection device of claim 1, wherein said sample collection device is made from polyethylene.

15. The sample collection device of claim 1, wherein said sample collection device is made from high density polyethylene.

16. A kit comprising:
a) a swab device comprising:
a swab end; and
a swab handle having a proximal end and a distal end, wherein said distal end comprises said swab end; and wherein said swab handle comprises a breakable junction between the swab end and the distal end of the swab handle;
b) a sample collection device comprising:
an extraction tube body comprising a dropper feature;
a top cap; and
a bottom cap; and
c) a buffer solution.

17-34. (canceled)

35. A method of collecting a sample, said method comprising:
a) providing the kit of claim 16;
b) adding at least a portion of the buffer solution to the extraction tube body to provide buffer solution in said extraction tube body;
c) contacting the swab device with a sample;
d) placing the swab device into the extraction tube body with the proximal end of said swab handle extending above a top end of the extraction tube body and the swab end contacting said buffer solution in said extraction tube body;
e) separating the swab end from at least a portion of the swab handle or compressing the swab device into the extraction tube;
f) securing the top cap to the extraction tube body;
g) removing a bottom cap from the extraction tube body; and
h) dispensing from the dropper feature buffer solution comprising at least a portion of the sample.

36-39. (canceled)
